(19) United States
(12) Patent Application Publication
(10) Pub. No.: US 2015/0272897 A1
(43) Pub. Date: Oct. 1, 2015

(54) COACERVATION ENCAPSULATION METHOD THAT DOES NOT INVOLVE THE USE OF TOXIC CROSS-LINKING AGENTS

(71) Applicant: SOCIETE D'EXPLOITATION DE PRODUITS POUR LES INDUSTRIES CHIMIQUES SEPPIC, Paris (FR)

(72) Inventor: Jean-Noel Ollagnier, Castres (FR)

(21) Appl. No.: 14/434,560

(22) PCT Filed: Oct. 8, 2013
(86) PCT No.: PCT/FR2013/052385

§ 371 Date: Apr. 9, 2015

(30) Foreign Application Priority Data
Oct. 9, 2012 (FR) 1259631

(51) Int. Cl. A61K 9/50; A61Q 19/00; A61K 8/65; A61K 8/73; A61K 8/49; A61K 8/11

(52) U.S. Cl. CPC A61K 9/5089; A61K 8/4953; A61K 8/11; A61K 8/65; A61K 8/733; A61Q 19/00; A61K 2800/594; A61K 2800/5426; A61K 2800/412; A61K 2800/5424

(57) ABSTRACT

A double-walled capsule includes a lipophilic core surrounded by a first layer of polymer coacervate and a second layer including a hydrogel, and which contains no trace of cross-linking agents.

The present invention relates to novel double-walled capsules obtained by coacervation without the use of a crosslinking agent and to the process for obtaining same, and also to the use of these capsules for preparing cosmetic compositions.

Coacervation describes the phenomenon of desolvation of macromolecules, such as polymers, resulting in a phase separation within a solution. Simple coacervation relates to processes involving the desolvation of a single polymer through one of the following factors: decrease in temperature, addition of a non-solvent, addition of electrolytes, addition of a second incompatible polymer. When the simultaneous desolvation of two water-soluble polyelectrolytes bearing opposite charges is caused by a modification of the pH of the aqueous medium, the term complex coacervation is used.

Complex coacervation is a well-known encapsulation technique which has been industrialized since the 1950s. It makes it possible to encapsulate water-insoluble ingredients. U.S. Pat. No. 2,800,457 of Jul. 23, 1957, describes for example the process for encapsulating oils in a coacervate of two organic polymers: gelatin and gum arabic. Canadian patent CA88263 of Oct. 5, 1971, describes a similar process using an organic polymer and an inorganic polymer.

The process for producing microcapsules by this technique is generally carried out in five successive steps.

In a first step, the product to be encapsulated (in liquid or solid form, pure or in oily solution) is dispersed in an aqueous solution containing two polymers having opposite charges (step a).

In a second step, the coacervation is induced by adjusting the pH of the solution, such that the positive charges of the first polymer cancel out the negative charges of the second (step b). The electrostatic attraction of the two polyelectrolytes causes the appearance of a mixed coacervate.

In a third step, the coacervate droplets formed are adsorbed (step c) at the surface of the active material to be encapsulated and form a continuous coating (step d).

In the fourth step, this coating is consolidated by crosslinking (step e) of the constituent macromolecules of the coacervate so as to form stable microcapsules.

Finally, the microcapsules are separated from the reaction medium by settling out and filtration, before undergoing washing or purifying operations in order to remove the unreacted products, in particular the excess crosslinking agents, and optionally drying operations.

Among the organic macromolecules or polymers of cationic nature that can be used in the coacervation technique, mention may be made, in a nonlimiting manner, of animal proteins such as pig or fish gelatin, albumin, vegetable proteins derived, for example, from soya, from potato or from wheat, chitosan and its derivatives, synthetic polymers resulting from the combining of amino acids, such as polylysine, or else polymers of vegetable origin such as guar gum and its derivatives.

Among the anionic organic polymers that may be used are natural polymers, such as gum arabic, alginates, carrageenates, cellulose derivatives such as carboxymethylcellulose, starch derivatives such as carboxymethyl starch, or synthetic polymers, such as acrylic, methacrylic, polylactic or polyglycolic polymers, or combinations thereof.

The ingredients encapsulated may be cosmetic active ingredients such as sunscreens, essential oils, vitamins A, D or E or their derivatives, or lipoamino acids.

In order to obtain capsules which are sufficiently mechanically strong, the crosslinking of step (e) is essential. This operation involves a crosslinking agent. Among the most effective crosslinking agents, mention may be made of formaldehyde or glutaraldehyde. Other crosslinking agents have also been proposed, such as carbodiimides, isocyanates (HDI or hexamethylene diisocyanate, TDI or toluene diisocyanate, IPDI or isopropyl diisocyanate), proanthocyanidins, etc. All these ingredients either have a not insignificant toxicity or are unstable in an aqueous medium and must be used under conditions which complicate the crosslinking step. Other authors have described cross-linking processes with enzymes, such as transglutaminases, or genepin. The costs of these products are such that only a few applications with very strong added values can be envisioned.

A crosslinking agent is a chemical compound which makes it possible to link one polymer chain to another via the formation of covalent bonds. In the prior art, it involves in particular a reaction between the aldehyde functions of the crosslinking agent and the residual amine functions of proteins, in particular with the amine functions of lysine units so as to form —N═CH-covalent bonds.

Glutaraldehyde is the crosslinking agent most commonly used. It is effective and inexpensive. However, it must be used at high doses, in the region of 1 to 5 mol/kg of gelatin (i.e. 100 to 500 g/kg of protein) and has a not insignificant toxicity both for the handler and for the user. Elimination of the excess glutaraldehyde is essential, in particular for all pharmaceutical, food or cosmetic applications; it requires numerous successive washes which consume water and are time consuming in order to obtain microcapsules containing an acceptable residual level of glutaraldehyde, below about one hundred ppm.

A first problem to be solved for the inventors of the present invention is therefore that of producing sufficiently strong microcapsules by means of a complex coacervation technique which does not use a crosslinking agent.

Given the very principle of the coacervation process, only lipophilic active agents, which are insoluble in water, may be incorporated into the microcapsules obtained by this technique. This undoubtedly constitutes a limitation of the process while a very large number of water-soluble ingredients must also be incorporated into the microcapsules.

An object of the invention is therefore to develop an improved coacervation technique, without the use of toxic or expensive crosslinking agents, which makes it possible to encapsulate both water-soluble and water-insoluble ingredients.

Other techniques have been described for encapsulating water-soluble ingredients, such as, for example, granulation by means of hydrophilic polymers, emulsification in oils in the form of water-in-oil emulsions, or absorption on ion exchange resins so as to form resinates. One of the techniques most widely used for encapsulating water-soluble ingredients remains incorporation into microbeads of water-soluble polymers such as chitosans, polyvinyl alcohols or alginates. Said polymers are used in numerous pharmaceutical or food applications for obtaining microbeads by means of a simple coacervation process, also called dripping. By way of example, the description of such a process for encapsulating cells will be found in patent application WO 91/09119 of Jun. 27, 1991.

Dripping consists in preparing an aqueous solution containing the water-soluble ingredient to be encapsulated and a polymer such as sodium alginate. This solution is pressurized through calibrated nozzles so as to form drops collected in an aqueous solution of divalent salts such as calcium chloride, magnesium chloride or manganese chloride. The calcium ions react with the sodium alginate so as to immediately form insoluble solid beads of calcium alginate. The beads obtained are separated by filtration or sieving and then generally washed with water so as to remove the excess calcium chloride.

In this context, the inventors of the present invention have developed novel double-walled capsules that may contain a lipophilic active agent in the primary capsules and optionally a hydrophilic active agent included in the second with a coacervation process without chemical crosslinking.

These original microcapsules consist of a lipophilic core surrounded by a first layer of polymer coacervate and a second layer comprising a hydrogel. They possess good tensile strength performance levels and stand out more particularly by virtue of their non-toxicity since no toxic crosslinking agent is used. They can also be modulated since it is possible to envision encapsulating both a lipophilic active agent in the core and a hydrophilic active agent in a hydrogel matrix. They may be provided either in wet form or in dry form with a reasonable cost price.

Thus, a subject of the invention is a process for preparing double-walled capsules comprising the following steps:

step a) dispersion of a lipophilic active ingredient in an aqueous solution, said solution containing at least one anionic polymer and at least one cationic polymer;
step b) adjustment of the pH of the solution obtained in step a) so that the positive charges of the cationic polymer cancel out the negative charges of the anionic polymer in order to induce a coacervation;
step c) adsorption of the coacervate droplets resulting from step b) at the surface of the active ingredient so as to form capsules;
step d) introduction of a solution of anionic polymers into the reaction medium containing the capsules obtained in step c);
step e) introduction of the mixture resulting from step d) into a means for forming drops;
step f) mixing of the drops resulting from step e) with a solution of divalent salts and formation of the double-walled capsules;
characterized in that no crosslinking agent is used.

According to other particular aspects, a subject of the invention is:
A process as described above, characterized in that said lipophilic active ingredient is chosen from sunscreens, essential oils, vitamins A, C, D or E or their derivatives, fragrances and aromas of natural origin, skin-tanning or -browning agents, N-acylated amino acid derivatives, such as N-palmitoyl alanine, N-palmitoyl glycine, N-palmitoyl leucine, N-palmitoyl isoleucine, N-cocoyl alanine or N-(ω-undecylenoyl) phenylalanine, ceramides, phospholipids and lipoamino acids.
A process as described above, characterized in that the anionic polymer is chosen from natural polymers, such as gum arabic, alginates, carrageenates, cellulose derivatives such as carboxymethylcellulose, starch derivatives such as carboxymethyl starch, or synthetic polymers, such as acrylic, methacrylic, polylactic or polyglycolic polymers, or combinations thereof.
A process as described above, characterized in that the cationic polymer is chosen from animal proteins such as pig or fish gelatin, albumin, vegetable proteins derived, for example, from soya, from potato or from wheat, chitosan and its derivatives, synthetic polymers resulting from the combining of amino acids, such as polylysine, or else polymers of vegetable origin such as guar gum and its derivatives.
A process as described above, characterized in that the solution of anionic polymers of step d) is a sodium alginate solution.
A process as described above, characterized in that said means for forming drops used in step e) is a nozzle or a needle.
A process as described above, characterized in that the solution of divalent salts of step f) is chosen from calcium chloride, barium chloride and manganese chloride solutions.
A process as described above, characterized in that the solution of anionic polymers of step d) contains a hydrophilic active ingredient to be encapsulated and, optionally, at least one additive chosen from finely divided insoluble solids of mineral origin, for instance silicas, laponites, aluminosilicates, titanium dioxide, or calcium sulfate, or of organic nature, for instance micronized waxes such as carnauba wax or beeswax, cationic polymers such as chitosan or polylysine, stearic acid or its micronized derivatives, microcrystalline cellulose, or starches.
A process as described above comprising step g) of filtration of the capsules obtained in step f); optionally a step h) of washing with water; and optionally a drying step.

The capsules may finally be dried by any drying process known to those skilled in the art, for instance in an oven, a lyophilizer or a fluidized bed. They may also be resuspended in an appropriate solution for being stored, transported and used in liquid form.

The invention also relates to a double-walled capsule comprising a lipophilic core surrounded by a first layer of polymer coacervate and a second layer comprising a hydrogel, characterized in that it contains no trace of crosslinking agent.

According to other particular aspects, a subject of the invention is:
A capsule as described above, characterized in that the lipophilic core comprises an active ingredient chosen from sunscreens, essential oils, vitamins A, C, D or E or their derivatives, fragrances and aromas of natural origin, skin-tanning or -browning agents, N-acylated amino acid derivatives, such as N-palmitoyl alanine, N-palmitoyl glycine, N-palmitoyl leucine, N-palmitoyl isoleucine, N-cocoyl alanine or N-(ω-undecylenoyl) phenylalanine, ceramides, phospholipids and lipoamino acids.
A capsule as described above, characterized in that the hydrogel comprises a hydrophilic active ingredient chosen from ingredients which show a soothing action on the skin, such as allantoin or bisabolol, ingredients which show a moisturizing action on the skin, for instance urea, hydroxyureas, glycerol, polyglycerols, glycerylglycosides and more particularly glycerol glucoside, xylitylglycosides and more particularly xylitylglucoside, polyphenol extracts, for instance grape polyphenol extracts, pine polyphenol extracts, wine polyphenol extracts, olive polyphenol extracts, plant extracts, for instance tannin-rich plant extracts, isoflavone-rich plant extracts, terpene-rich plant extracts, extracts of freshwater or seawater algae, bacterial extracts, ingredients which show an antimicrobial action or a purifying action.
A capsule as described above, having a diameter of between 100 μm and 3000 μm and preferably between 500 μm and 2000 μm.
A capsule as described above, capable of being obtained by means of the process as defined above.
A capsule as described above, characterized in that it comprises from 0.5% to 40% by weight of lipophilic active agent, more particularly from 1% to 30%, and even more particularly from 1% to 20%, from 0% to 20% by weight of hydrophilic active agent, more particularly from 0.5% to 10% and even more particularly from 0.5% to 5%, and from 0.1% to 5% by weight of anionic polymer, more particularly from 0.5% to 5%, and even more particularly from 1% to 3%.

Finally, a subject of the invention is the use of at least one capsule as defined above, for preparing a cosmetic composition. A subject of the invention is also a cosmetic composition comprising from 0.01% to 20% by weight, more particularly from 1% to 10% by weight of at least one capsule according to the invention. With regard to the cosmetic compositions comprising the capsules, they may be in the form of O/W, W/O, W/O/W, O/W/O emulsions and/or aqueous gels with at least one hydrophilic-phase thickener.

Moreover, the cosmetic compositions which are in the form of emulsions may comprise an oil phase which is thick because of the presence of oil-thickening polymers such as those described in FR2961513A1 and in FR2961210A1.

The encapsulated ingredients may be cosmetic active ingredients such as:

a) sunscreens. The sunscreens may be:
1) organic sunscreens chosen from organic screening agents which are active in the UV-A range, organic screening agents which are active in the UV-B range, organic screening agents which are active in the UV-A and UV-B ranges, and mixtures thereof.

Among the organic screening agents which are active in the UV-A range, mention may be made of:
dibenzoylmethane derivatives, for example the butylmethoxydibenzoylmethane or azobenzone sold under the brand name Parsol™ 1789,
the menthyl anthranilate sold under the brand name Neo Heliopan™ MA by the company Symrise,
and mixtures thereof.

Among the screening agents which are active only in the UV-B range, mention may be made of:
salicylic derivatives, such as the homosalate sold under the brand name Neo Heliopan™ HMS; the ethylhexyl salicylate sold under the brand name Neo Heliopan™ OS by Symrise; the octyl salicylate sold under the brand name Neo Heliopan® type 05;
cinnamates, for instance sold under the brand name Parsol™ MCX by the company DSM, isopropyl ethoxy-cinnamate, the isoamyl methoxycinnamate sold under the brand name Neo Heliopan™ E 1000 by Symrise, diisopropyl methyl cinnamate, cinnoxate, glycerylethylhexanoate dimethoxycinnamate;
benzylidenecamphor derivatives, for instance 3-benzylidene, the methylbenzylidenecamphor sold under the brand name Neo Heliopan® MBC;
triazine derivatives, for instance the ethylhexyl-triazone sold under the brand name Uvinul™ T150 by BASF, the diethylhexylbutamidotriazone sold under the trade name Uvasorb™ HEB, bis-ethylhexyloxyphenol methoxyphenyl triazine;
para-aminobenzoates (or PABAs), for instance ethyl PABA, ethyldihydroxypropyl PABA, the ethylhexyldimethyl PABA sold under the brand name Escalol™ 507 by the company ISP;
imidazoline derivatives, for instance ethylhexyl-dimethoxybenzylidenedioxoimidazoline propionate;
benzalmalonate derivatives, for instance polyorganosiloxanes comprising a benzalmalonate function, such as the polysilicone-15 sold under the brand name Parsol® SLX by the company DSM, dineopentyl 4′-methoxybenzalmalonate.

Among the screening agents which are active both in the UV-A range and in the UV-B range, mention may be made of:
benzophenone derivatives, for instance the benzophenone-1 sold under the brand name Uvinul™ 400, the benzophenone-2 sold under the brand name Uvinul™ D50, the benzophenone-3 or oxybenzone sold under the brand name Uvinul™ M40, the benzophenone-4 sold under the brand name Uvinul™ MS40, the benzophenone-6 sold under the brand name Helisorb™ 11, the benzophenone-8 sold under the brand name Spectrasorb™ UV-24,
phenylbenzotriazole derivatives, for instance the drometrizole trisiloxane sold under the brand name Silatrizole™ by the company Rhodia, the methylene bis-benzotriazolyl tetramethylbutylphenol sold under the brand name Mixxim™ BB/100,
bis-resorcinyltriazine derivatives, for instance bis-ethylhexyloxyphenol methoxyphenyl triazine,
benzoxazole derivatives, for instance the 2,4-bis-[4-[5-(1,1-dimethyl-propyl)benzoxazol-2-yl]phenylimino]-6-[(2-ethylhexyl)imino]-1,3,5-triazine sold under the brand name Uvasorb™ K2A by the company Sigma 3V;
and mixtures thereof;
   2) inorganic sunscreens chosen from talc, kaolin, treated or untreated metal oxide pigments, for instance nanopigments of titanium oxide (amorphous or crystalline), of iron oxide, of zinc oxide, of zirconium oxide or of cerium oxide;
      b) essential oils, for instance:
essential oils rich in terpene and sesquiterpene carbon-based compounds, such as turpentine essential oil, juniper essential oil, lemon essential oil,
alcohol-rich essential oils, such as coriander essential oil, rose essential oil, rosewood essential oil,
essential oils rich in mixtures of esters and of alcohols, such as lavender essential oil, mint essential oil,
aldehyde-rich essential oils, such as cinnamon essential oil, citronella essential oil, eucalyptus essential oil,
ketone-rich essential oils, such as sage essential oil, camphor tree essential oil, thuja essential oil,
phenol-rich essential oils, such as thyme essential oil, savory essential oil, oregano essential oil, clove essential oil,
ether-rich essential oils, such as star anise essential oil, aniseed essential oil, fennel essential oil, Eucalyptus globulus essential oil,
peroxide-rich essential oils, such as Chenopodium essential oil, garlic essential oil,
sulfurous essential oils, such as essential oils of cruciferae and of liliaceae;
   c) fragrancing substances, for instance:
a. essential oils,
b. fragrances and aromas of natural origin, such as extracts of flowers, of stalks, of leaves, of fruits, of roots, of wood, of needles, of branches, of herbs, of grasses, of resins, of balms,
c. substances of synthetic origin, such as:
i. compounds of ester type, for instance benzyl acetate, benzyl benzoate, phenoxyethyl isobutyrate, p-tert-butylcyclohexyl acetate, citronellyl acetate, citronellyl formate, geranyl acetate, linalyl acetate, dimethyl-benzylcarbinyl acetate, phenylethyl acetate, linalyl benzoate, benzyl formate, ethyl-methylphenyl glycinate, alkylcyclohexyl propionate, styralyl propionate and benzyl salicylate,
ii. compounds of ether type, for instance benzyl ethyl ether,
iii. compounds of aldehyde type, for instance citral, citronellal, citronellyloxy-acetaldehyde, cyclamenaldehyde, hydroxy-citronellal, lilial and bourgeonal,
iv. compounds of ketone type, for instance ionones and more particularly α-isomethyl-ionone,
v. compounds of aromatic alcohol type, for instance anethole, citronellol, eugenol, isoeugenol, geraniol, linalol, phenylethyl alcohol, terpineol,
vi. terpenes;
   d) skin-tanning or -browning agents, for instance dihydroxyacetone, isatin, alloxan, ninhydrin, erythrulose;
   e) dyes, for instance Yellow 5, Acid Blue 91, Green 5, Green 3/Fast Green FCF 3, Orange 4, Red 4/Food Red 1, Yellow 6, Acid Red 33/Food Red 12, Red 40, cochineal carmine (CI 15850, CI 75470), Ext. Violet 2, Red 6-7, ferric ferrocyanide, ultramarines, Acid Yellow 3/Yellow 10, Acid Blue 3, Yellow 10;
   f) vitamins or derivatives thereof, for instance retinol (vitamin A) and esters thereof (retinyl palmitate for example), ascorbic acid (vitamin C) and esters thereof, ascorbic acid sugar derivatives (for instance ascorbyl glucoside), tocopherol (vitamin E) and esters thereof (for instance tocopheryl acetate), vitamins B3 or B10 (niacinamide and derivatives thereof);
   g) amino acids;
   h) total or partial protein hydrolysates;
   i) N-acylated amino acid derivatives of formula (A):

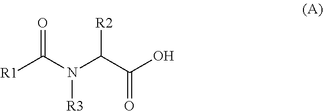

in which:
R1 represents a linear or branched, saturated or unsaturated, aliphatic radical comprising from 7 to 29 carbon atoms,
R2 represents a hydrogen atom or a radical chosen from methyl, isopropyl, isobutyl, 1-methyl-propyl, hydroxymethyl, 1-hydroxyethyl, thiomethyl, 2-methylthioethyl, 4-aminobutyl, 3-guanidinopropyl, 3-ureidopropyl, (1-aminocarbonyl)methyl, carboxymethyl, 2-carboxyethyl, 2-(aminocarbonyl)ethyl, benzyl, 4-hydroxybenzyl, 3,4-dihydroxybenzyl, [1H-indol-3-yl]-methyl, (1H-imidazol-4-yl)methyl and 3-aminopropyl radicals, and
R3 represents a hydrogen atom or a methyl radical.

As examples of N-acylated amino acid derivatives of formula (A), mention may be made of N-palmitoyl alanine, N-palmitoyl glycine, N-palmitoyl leucine, N-palmitoyl isoleucine, N-cocoyl alanine and N-(ω-undecylenoyl) phenylalanine;

j) N-acylated amino acid derivatives of formula (B)

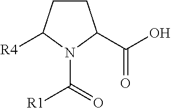

in which
R1 represents a linear or branched, saturated or unsaturated, aliphatic radical comprising from 7 to 29 carbon atoms, and
R4 represents a hydrogen atom or a hydroxyl radical;
   k) ingredients which show a skin lightening and/or bleaching and/or discoloring action, for instance arbutin, kojic acid, hydroquinone or its derivatives, Vegewhite™, Gatuline™, Synerlight™, Biowhite™, Phytolight™, Dermalight™, Clariskin™, Melaslow™, Dermawhite™, Ethioline, Melarest™, Gigawhite™, Albatine™, Lumiskin™;
   l) ingredients which show a soothing action on the skin, such as allantoin or bisabolol;
   m) ingredients which show a skin-moisturizing action, for instance urea, hydroxyureas, glycerol, polyglycerols, glycerylglycosides and more particularly glycerol glucoside, xylitylglycosides and more particularly xylitylglucoside, contained in the composition sold under the brand name Aquaxyl™;
   n) polyphenol extracts, for instance grape polyphenol extracts, pine polyphenol extracts, wine polyphenol extracts, olive polyphenol extracts;
   o) plant extracts, for instance tannin-rich plant extracts, isoflavone-rich plant extracts, terpene-rich plant extracts;
   p) extracts of freshwater or seawater algae;
   q) bacterial extracts;
   r) ceramides;
   s) phospholipids;
   t) ingredients which show an antimicrobial action or a purifying action, for instance Octopirox™ or Sensiva™ SC50.

Surprisingly, the non-crosslinked microcapsules obtained in step (d) are stable in the presence of the anionic polymer solution added and do not break when steps (e) and (f) of the process according to the invention are carried out. The lipophilic ingredient remains confined in the oily core of the novel microcapsule and the hydrophilic ingredient is encapsulated in the second alginate shell.

The anionic polymer solution used in step (e) may also contain technological additives intended to reinforce the mechanical strength of the microbeads, to adjust their density or to modulate the hydrophilic ingredient release kinetics. These additives may be finely divided insoluble solids of mineral nature, for instance silicas, laponites, aluminosilicates, titanium dioxide or calcium sulfate, or of organic nature, such as micronized waxes (carnauba wax, beeswax, etc.), cationic polymers such as chitosan or polylysine, stearic acid or its micronized derivatives, microcrystalline cellulose, or starches. The technological additives may also be soluble products such as mineral salts, glycols or surfactants which allow better dispersion of the microcapsules or which facilitate the drying operations.

The following examples describe an implementation of the process according to the invention and the microcapsules obtained.

### EXAMPLE NO. 1

### Protocol for double encapsulation by coacervation with potato protein and gum arabic then insertion into alginate/laponite beads

| Material required | Products required |
|---|---|
| Glass reactor (2 l) | 10 g of potato isolate |
| Stirring paddle | 15 g of gum arabic |
| Heating bath | 625 g of demineralized water |
| Beakers | 100 g of MCT (C₈-C₁₀ triglyceride) oil |
| pH probe | 0.1 g of red oil dye |
| Magnetic stirrer | 10% acetic acid |
| 100 μm filter | 4 g of powdered alginate |
| Syringe | 1 g of laponite |
| | Solution [CaCl₂] = 4% |

1.1 Preparation of microcapsules by coacervation

Oil Phase

0.1 g of red oil and 100 g of MCT oil are placed in a beaker and stirred with a magnetic stirrer for 20 minutes at 40° C. Filtration is carried out and the resulting product is left to cool.

Aqueous Phase

15 g of gum arabic are placed in a beaker containing 400 g of water. Stirring is carried out with a magnetic stirrer until dissolution is obtained (5 minutes), then the mixture is placed in the reactor and stirring is carried out at 200 revolutions per minute (rpm).

Incorporation of the Oil Phase

The stirring of the reactor is increased to 350 rpm and then the oil is slowly introduced. The resulting mixture is left to stir for 15 minutes.

Potato Isolate

10 g of potato isolate and 225 g of water are placed in a beaker and stirred with a magnetic stirrer. When dissolution is complete (5 minutes) the solution is very slowly introduced into the reactor while controlling the pH (approximately 4 after the entire addition).

Lowering of the pH

The pH of the medium is reduced to 3.65 with 10% acetic acid so that the coacervation forms.

Increase in Temperature

The temperature is increased to 50° C. for 1 hour in order to harden the potato isolate. The resulting product is left to cool and to settle out.

1.2 Preparation of microbeads by double encapsulation

Preparation of the Alginate Solution

4 g of alginate and 1 g of laponite are slowly placed in a beaker containing 200 g of water with vigorous stirring for 30 minutes.

Microcapsules/Alginate Mixture

100 g of microcapsules obtained in step 1.1 are weighed out and placed in 150 g of a 2% sodium alginate solution.

Microbead Formation

The mixture is placed in a syringe and drops are made in the calcium chloride solution. They are left for a contact time of 15 minutes. To finish, they are rinsed with water.

1.3 Characterization of the microbeads obtained by means of the process

The non-dried beads obtained by means of the process are colored spheres having an average size of 1000 μm.

They contain approximately 20% of an oily core, 5% of a gelatin/gum arabic coacervate, 1% of alginate and 0.5% of laponite, the remainder being water.

When a mechanical pressure is exerted on the microbeads, they burst, releasing red oil.

### EXAMPLE NO. 2

### Protocol for Double Encapsulation by Coacervation with Gelatin and Gum Arabic then Insertion into Alginate Beads

2.1 Preparation of the microcapsules by coacervation

| Material required | Products required |
|---|---|
| Glass reactor (2 l) | 12.5 g of gelatin |
| Stirring paddle | 12.5 g of gum arabic |
| Heating bath | 550 g of demineralized water |
| Ice bath | 100 g of MCT oil |
| Beakers | 0.1 g of red oil dye |
| pH probe | 10% acetic acid |
| Thermometer | 4 g of powdered alginate |
| Magnetic stirrer | 1 g of laponite |
| 100 μm filter | Solution [CaCl₂] = 4% |
| Syringe | |

Oil Phase

0.1 g of red oil (used as model lipophilic ingredient to be encapsulated) and 100 g of MCT oil are placed in a beaker and stirred with a magnetic stirrer for 20 minutes at 40° C. The resulting product is filtered and left at 40° C.

Aqueous Phase

300 g of water are placed in the reactor thermostatted at 40° C.

12.5 g of gum arabic, and 12.5 g of gelatin are placed in a beaker containing 250 g of water. Stirring is carried out at 40° C. until dissolution is obtained (15 minutes). The mixture is then added to the reactor and stirred at 200 rpm.

Incorporation of the Oil Phase

The stirring is increased to 350 rpm and then the hot oil is slowly introduced. It is left to stir for 15 minutes.

### Lowering of the pH

The heating is stopped and the pH of the medium is reduced to 4.10 with 10% acetic acid so that the coacervation forms.

Lowering of the Temperature

The temperature is reduced to 10° C. in order to harden the gelatin. The resulting product is left for 15 minutes and then the stirring is stopped. The resulting product is then left to settle out.

2.2 Preparation of microbeads by double encapsulation

Preparation of the Alginate Solution

4 g of alginate and 1 g of laponite are slowly placed in a beaker containing 200 g of water with vigorous stirring for 30 minutes.

Microcapsules/Alginate (50/50) Mixture

100 g of microcapsules are weighed out and placed in the same amount of sodium alginate solution. Homogenization is carried out with magnetic stirring.

The mixture is placed in a syringe and drops are made in the calcium chloride solution. They are left for a contact time of 15 minutes, and then rinsed with water.

### 2.3 Characterization of the Microbeads Obtained by Means of the Process

The microbeads obtained are translucent, having an average size of approximately 800 μm. The primary microcapsules of oil and gelatin are clearly visible inside the microbeads.

The microbeads contain approximately 20% of an oily core, 5% of a gelatin/gum arabic coacervate, 1% of alginate and 0.5% of laponite, the remainder being water.

It was verified that the microbeads according to the invention are stable and leaktight. For this, they were dispersed in a mineral oil and subjected to magnetic stirring for two hours. The coloration of the mineral oil was observed after two hours. Any coloration of this oil reflects diffusion of the dye and rupture of the microcapsules. By way of control, microcapsules produced by means of the coacervation process without crosslinking, obtained as described in paragraph 2.1, and microbeads obtained by the same process but crosslinked with glutaraldehyde were subjected to the same test.

| | | | Microbeads |
|---|---|---|---|
| | Non- | Glutaraldehyde- | according |
| | crosslinked | crosslinked | to the |
| | microcapsules | microcapsules | invention |
| Coloration | Bright red | No coloration | No coloration |
| of the oil | | | |
| after 2 h | | | |

These results demonstrate the good stability of the microbeads according to the invention.

### EXAMPLE NO. 3

### Protocol for Double Encapsulation of the Microcapsules (by Coacervation with Gelatin) in Alginate Beads

3.1 Preparation of the microcapsules by coacervation

| Material required | Products required |
|---|---|
| Glass reactor (2 l) | 12.5 g of gelatin |
| Stirring paddle | 12.5 g of gum arabic |
| Heating bath | 550 g of demineralized water |
| Ice bath | 100 g of MCT oil |
| Beakers | 0.1 g of red oil dye |
| pH probe | 10% acetic acid |
| Thermometer | 4 g of powdered alginate |
| Magnetic stirrer | 1 g of caffeine |
| 100 μm filter | Solution [CaCl₂] = 4% |
| NISCO VAR D drop generator | |

The same protocol as that described in example 2 is carried out.

3.2 Preparation of microbeads by double encapsulation

Preparation of the Solution of Alginate and Caffeine

4 g of alginate and 1 g of caffeine are slowly placed in a beaker containing 200 g of water with vigorous stirring for 30 minutes.

Microcapsules/Alginate (50/50) Mixture

100 g of microbeads obtained in step 3.1 are weighed out and the same amount of 2% alginate is introduced therein. Mixing is carried out in order to obtain a homogeneous suspension.

The mixture is introduced into the reservoir of the NISCO VAR D drop generator equipped with a nozzle having a diameter of 800 μm and the apparatus is started with the following parameters:
Flow rate: 12 ml/min, frequency of vibrations of the vibrating nozzle: 0.22 kHz, amplitude 79%.

The drops generated are harvested in the calcium chloride solution where they form solid microbeads. They are left for a contact time of 15 minutes, and the beads are filtered and rinsed with water.

### 3.3 Characterization of the Microbeads Obtained by Means of the Process

Microbeads having an average diameter of 1500 μm are obtained, containing two encapsulated ingredients: caffeine in the external alginate matrix and red oil in the oily core. Their quantitative composition is approximately 20% of oil, 5% of a gelatin/gum arabic coacervate, 1% of alginate or 0.5% of caffeine, the remainder being water.

1. A process for preparing double-walled capsules comprising the following steps:
step a) dispersion of a lipophilic active ingredient in an aqueous solution, said solution containing at least one anionic polymer and at least one cationic polymer;
step b) adjustment of the pH of the solution obtained in step a) so that the positive charges of the cationic polymer cancel out the negative charges of the anionic polymer in order to induce a coacervation;
step c) adsorption of the coacervate droplets resulting from step b) at the surface of the active ingredient so as to form capsules;
step d) introduction of a solution of anionic polymers into the reaction medium containing the capsules obtained in step c);
step e) introduction of the mixture resulting from step d) into a means for forming drops;
step f) mixing of the drops resulting from step e) with a solution of divalent salts and formation of the double-walled capsules;
wherein no crosslinking agent linking one polymer chain to another via the formation of covalent bonds is used.

2. The process as claimed in claim 1, wherein said lipophilic active ingredient is chosen from sunscreens, essential oils, vitamins A, C, D or E or their derivatives, fragrances and aromas of natural origin, skin-tanning or -browning agents, N-acylated amino acid derivatives, such as N-palmitoyl alanine, N-palmitoyl glycine, N-palmitoyl leucine, N-palmitoyl isoleucine, N-cocoyl alanine or N-(ω-undecylenoyl) phenylalanine, ceramides, phospholipids and lipoamino acids.

3. The process as claimed in claim 1, wherein the anionic polymer is chosen from natural polymers, such as gum arabic, alginates, carrageenates, cellulose derivatives such as carboxymethylcellulose, starch derivatives such as carboxymethyl starch, derivatives such as carboxymethyl starch, or synthetic polymers, such as acrylic, methacrylic, polylactic or polyglycolic polymers, or combinations thereof.

4. The process as claimed in claim 1, wherein the cationic polymer is chosen from animal proteins such as pig or fish gelatin, albumin, vegetable proteins derived, for example, from soya, from potato or from wheat, chitosan and its derivatives, synthetic polymers resulting from the combining of amino acids, such as polylysine, or else polymers of vegetable origin such a guar gum and its derivatives.

5. The process as claimed in claim 1, wherein the solution of anionic polymers of step d) is a sodium alginate solution.

6. The process as claimed in claim 1, wherein said means for forming the drops used in step e) is a nozzle or a needle.

7. The process as claimed in claim 1, wherein the solution of divalent salts of step f) is chosen from calcium chloride, barium chloride and manganese chloride solutions.

8. The process as claimed in claim 1, wherein the solution of anionic polymers of step d) contains a hydrophilic active ingredient to be encapsulated and, optionally, at least one additive chosen from finely divided insoluble solids of mineral nature, for instance silicas, laponites, aluminosilicates, titanium dioxide or calcium sulfate, or of organic nature, for instance micronized waxes such as carnauba wax or beeswax, cationic polymers such as chitosan or polylysine, stearic acid or its micronized derivatives, microcrystalline cellulose, or starches.

9. The process as claimed in claim 1, further comprising step g) of filtration of the capsules obtained in step f); optionally a step h) of washing with water; optionally a drying step.

10. A double-walled capsule comprising a lipophilic core surrounded by a first layer of polymer coacervate and a second layer comprising a hydrogel, and which contains no trace of crosslinking agent.

11. The capsule as claimed in claim 10, wherein the lipophilic core comprises an active ingredient chosen from sunscreens, essential oils, vitamins A, C, D or E or their derivatives, fragrances and aromas of natural origin, skin-tanning or -bronzing agents, N-acylated amino acid derivatives, such as N-palmitoyl alanine, N-palmitoyl glycine, N-palmitoyl leucine, N-palmitoyl isoleucine, N-cocoyl alanine or N-(ω-undecylenoyl) phenylalanine, ceramides, phospholipids and lipoamino acids.

12. The capsule as claimed in claim 10, wherein the hydrogel comprises a hydrophilic active ingredient chosen from ingredients which show a soothing action on the skin, such as allantoin or bisabolol, ingredients which show a moisturizing action on the skin, for instance urea, hydroxyureas, glycerol, polyglycerols, glycerylglycosides and more particularly glycerol glucoside, xylitylglycosides and more particularly xylitylglucoside, polyphenol extracts, for instance grape polyphenol extracts, pine polyphenol extracts, wine polyphenol extracts, olive polyphenol extracts, plant extracts, for instance tannin-rich plant extracts, isoflavone-rich plant extracts, terpene-rich plant extracts, extracts of freshwater or seawater algae, bacterial extracts, ingredients which show an antimicrobial action or a purifying action.

13. The capsule as claimed in claim 10, having a diameter of between 100 μm and 3000 μm and preferably between 500 μm and 2000 μm.

14. The capsule obtained by the process as defined in claim 1, comprising a lipophilic core surrounded by a first layer of polymer coacervate and a second layer comprising a hydrogel, and which contains no trace of crosslinking agent.

15. The capsule as claimed in claim 10, further comprising from 0.5% to 40% by weight of lipophilic active agent, more particularly from 1% to 30%, and even more particularly from 1% to 20%, from 0% to 20% by weight of hydrophilic active agent, more particularly from 0.5% to 10% and even more particularly from 0.5% to 5%, and from 0.1% to 5% by weight of anionic polymer, more particularly from 0.5% to 5%, and even more particularly from 1% to 3%.

16. (canceled)

17. A cosmetic composition comprising from 0.01% to 20% by weight, more particularly from 1% to 10% by weight of at least one capsule as defined in claim 10.
